(19) United States
(12) Patent Application Publication
(10) Pub. No.: US 2019/0269721 A1
(43) Pub. Date: Sep. 5, 2019

(54) METHODS AND COMPOSITIONS FOR DEGRADING CALCIUM PHOSPHATE AND FOR TREATING CALCIFICATION DISORDERS

(71) Applicant: MERZ NORTH AMERICA, INC., Raleigh, NC (US)

(72) Inventor: Deanne Mraz ROBINSON, Ridgefield, CT (US)

(73) Assignee: MERZ NORTH AMERICA, INC., Raleigh, NC (US)

(21) Appl. No.: 16/344,738

(22) PCT Filed: Nov. 6, 2017
(86) PCT No.: PCT/US2017/060102

§ 371 Date: Apr. 24, 2019

Related U.S. Application Data
(60) Provisional application No. 62/566,721, filed on Oct. 2, 2017.
(60) Provisional application No. 62/417,882, filed on Nov. 4, 2016.

(51) Int. Cl. A61K 33/04; A61K 9/00; A61P 17/00

(52) U.S. Cl. CPC A61K 33/04; A61P 17/00; A61K 9/0019; A61K 9/0014

(57) ABSTRACT

The present disclosure provides for methods and compositions for degrading, dissolving or reducing the level of tissue filler implanted in a subject. The pharmaceutical composition contains a thiosulfate, a meta bisulfite, or a combination thereof. Also encompassed by the present disclosure are methods and compositions for treating or preventing a calcification disorder.

### 1. CROSS REFERENCE TO RELATED APPLICATION

This application claims priority to U.S. Provisional Application Nos. 62/417,882, filed Nov. 4, 2016 and 62/566,721, filed Oct. 2, 2017, both of which are incorporated by reference herein in their entireties.

### 2. INTRODUCTION

The present disclosure relates to methods and compositions for reducing the amount of tissue fillers implanted in a patient, and for treating or preventing a calcification disorder. In particular, the present disclosure relates to using a thiosulfate and/or a metabisulfite to reduce the amount of calcium hydroxyapatite tissue fillers, and for treating a calcification disorder.

### 3. BACKGROUND

Radiesse® (Merz North America, Inc.) is a calcium hydroxyapatite-based soft tissue filler. It is approved by the FDA for hand augmentation and for treatment of moderate to severe facial wrinkles. Currently, concerns exist regarding the use of Radiesse® due to the lack of an effective agent that can metabolize calcium hydroxyapatite. The irreversibility of Radiesse® treatment raises safety issues if inadvertent intravascular injection, overcorrection, vascular compromise, or nodule formation occurs. In fact, severe complications have been reported with Radiesse® use (Carruthers et al., Plast. Reconstr. Surg. 2014 December; 134(6): 1197-201. Blindness caused by cosmetic filler injection: a review of cause and therapy.). Moreover, it is now believed that the permanent impact of filler materials in tissue is undesired, since the tissue itself is subject to ageing. E. Haneke, Skin rejuvenation without a scalpel I. Fillers, J. Cosmetic Dermatology, 5: 15-167 (2005). K. De Boulle, Management of complications after implantation of fillers. J. Cosmetic Dermatology. 3:2-15 (2004).

Therefore, despite its superior longevity and volume replacement, medical providers and patients have been apprehensive to use Radiesse®. Trials of using various agents to dissolve Radiesse® have been largely unsuccessful. For example, hyaluronidase, which dissolves hyaluronic acid based fillers, cannot effectively dissolve calcium hydroxyapatite. To date, no agents have been shown to be able to dissolve calcium hydroxyapatite (CaHA) filler after placement. Additionally, chelating agents targeting divalent cations, such as ethylenediaminetetraacetic acid (EDTA), has also been unsuccessful in reducing the amount of calcium hydroxyapatite.

There is a range of cutaneous calcification disorders, either from metastatic or systemic disease, or primarily involving skin. There are case reports of off-label use of topical sodium metabisulfite (SMB) in the treatment of calcinosis cutis (Barrio-Díaz et al., Topical sodium metabisulfite for the treatment of calcinosis cutis: A promising new therapy. Br. J. Dermatol. 2016, 175(3):608-11). Additionally, there has been treatment with intravenous and/or intralesional sodium thiosulfate (STS) for cutaneous calciphylaxis (Strazzula et al., Intralesional sodium thiosulfate for the treatment of calciphylaxis, JAMA Dermatol. 2013 August; 149(8): 946-9). However, there is still a need to effectively degrade or dissolve calcium phosphate tissue fillers, and to treat or prevent calcification disorders.

### 4. SUMMARY

Provided herein is a method of reducing the amount of calcium phosphate tissue filler implanted in a subject, comprising the step of administering to the subject an effective amount of a pharmaceutical composition comprising a thiosulfate, a metabisulfite, or a combination thereof.

In one embodiment, the thiosulfate is sodium thiosulfate (STS).

In one embodiment, the pharmaceutical composition (or medicament) is administered to the subject through intralesional injection.

In one embodiment, the pharmaceutical composition comprises about 25% (w/v) sodium thiosulfate.

In one embodiment, the metabisulfite is sodium metabisulfite (SMB).

In one embodiment, the pharmaceutical composition comprises about 25% (w/v) sodium metabisulfite.

In one embodiment, the pharmaceutical composition (or medicament) is administered topically to the subject.

In one embodiment, the calcium phosphate is calcium hydroxyapatite.

In one embodiment, the volume ratio of the tissue filler to the pharmaceutical composition ranges from about 4:1 to about 1:3.

In one embodiment, the volume ratio of the tissue filler to the pharmaceutical composition is about 1:1, 1:2 or 1:2.5.

In one embodiment, the amount of the calcium phosphate tissue filler is reduced at least 50% about 24 hours after administration of the pharmaceutical composition.

In one embodiment, the amount of the calcium phosphate tissue filler is reduced at least 80% about 24 hours after administration of the pharmaceutical composition.

In one embodiment, the amount of the calcium phosphate tissue filler is reduced at least 90% about 24 hours after administration of the pharmaceutical composition.

In one embodiment, the calcium phosphate tissue filler is implanted into the subject in a plastic and/or reconstructive surgery.

In one embodiment, the calcium phosphate tissue filler is for hand augmentation.

In one embodiment, the calcium phosphate tissue filler is for hard or soft tissue augmentation, regeneration, and/or repair.

In one embodiment, the calcium phosphate tissue filler is for skin contour deficiencies.

In one embodiment, the skin contour deficiencies comprise frown lines, worry lines, wrinkles, crow's feet, marionette lines, stretch marks, scars, or combinations thereof.

In one embodiment, the subject is a human.

Also provided is a method of treating a calcification disorder in a subject, comprising the step of administering to the subject an effective amount of a pharmaceutical composition comprising a thiosulfate, a metabisulfite, or a combination thereof.

In one embodiment, the thiosulfate is sodium thiosulfate (STS).

In one embodiment, the pharmaceutical composition comprises about 25% (w/v) sodium thiosulfate.

In one embodiment, the metabisulfite is sodium metabisulfite (SMB).

In one embodiment, the pharmaceutical composition comprises about 25% (w/v) sodium metabisulfite.

In one embodiment, the pharmaceutical composition is administered topically.

In one embodiment, the pharmaceutical composition is administered systemically.

In one embodiment, the calcification disorder is a cutaneous calcification or a subcutaneous calcification.

In one embodiment, the calcification disorder is calcium hydroxyapatite depositions resulting from implantation of calcium phosphate tissue filler.

In one embodiment, the calcification disorder is a systemic calcium hydroxyapatite deposition disease.

In one embodiment, the calcification disorder is a cardiovascular calcification.

The present disclosure provides for use of a thiosulfate, a metabisulfite, or a combination thereof, in the manufacture of a medicament for reducing an amount of calcium phosphate tissue filler implanted in a subject.

The present disclosure provides for use of a thiosulfate, a metabisulfite, or a combination thereof, in the manufacture of a medicament for treating a calcification disorder in a subject.

In one embodiment, the thiosulfate is sodium thiosulfate (STS).

In one embodiment, the pharmaceutical composition (or medicament) is administered to the subject through intralesional injection.

In one embodiment, the pharmaceutical composition (or medicament) comprises about 25% (w/v) sodium thiosulfate.

In one embodiment, the metabisulfite is sodium metabisulfite (SMB).

In one embodiment, the pharmaceutical composition (or medicament) comprises about 25% (w/v) sodium metabisulfite.

In one embodiment, the pharmaceutical composition (or medicament) is administered topically to the subject.

In one embodiment, the calcium phosphate is calcium hydroxyapatite.

In one embodiment, the volume ratio of the tissue filler to the pharmaceutical composition ranges from about 4:1 to about 1:3.

In one embodiment, the volume ratio of the tissue filler to the pharmaceutical composition is about 1:1, 1:2 or 1:2.5.

In one embodiment, the amount of the calcium phosphate tissue filler is reduced at least 50% about 24 hours after administration of the pharmaceutical composition (or medicament).

In one embodiment, the amount of the calcium phosphate tissue filler is reduced at least 80% about 24 hours after administration of the pharmaceutical composition (or medicament).

In one embodiment, the amount of the calcium phosphate tissue filler is reduced at least 90% about 24 hours after administration of the pharmaceutical composition (or medicament).

In one embodiment, the calcium phosphate tissue filler is implanted into the subject in a plastic and/or reconstructive surgery.

In one embodiment, the calcium phosphate tissue filler is for hand augmentation.

In one embodiment, the calcium phosphate tissue filler is for hard or soft tissue augmentation, regeneration, and/or repair.

In one embodiment, the calcium phosphate tissue filler is for skin contour deficiencies.

In one embodiment, the skin contour deficiencies comprise frown lines, worry lines, wrinkles, crow's feet, marionette lines, stretch marks, scars, or combinations thereof.

In one embodiment, the subject is a human.

The present disclosure provides for a pharmaceutical composition comprising a thiosulfate, a metabisulfite, or a combination thereof, for use in reducing an amount of calcium phosphate tissue filler implanted in a subject.

The present disclosure provides for a pharmaceutical composition comprising a thiosulfate, a metabisulfite, or a combination thereof, for use in treating a calcification disorder in a subject.

In one embodiment, the thiosulfate is sodium thiosulfate (STS).

In one embodiment, the pharmaceutical composition (or medicament) is administered to the subject through intralesional injection.

In one embodiment, the pharmaceutical composition (or medicament) comprises about 25% (w/v) sodium thiosulfate.

In one embodiment, the metabisulfite is sodium metabisulfite (SMB).

In one embodiment, the pharmaceutical composition (or medicament) comprises about 25% (w/v) sodium metabisulfite.

In one embodiment, the pharmaceutical composition (or medicament) is administered topically to the subject.

The present disclosure provides for a pharmaceutical combination, preferably in a kit, comprising: (a) a calcium phosphate tissue filler, and (b) a pharmaceutical composition comprising a thiosulfate, a metabisulfite, or combination thereof.

In one embodiment, the thiosulfate is sodium thiosulfate (STS).

In one embodiment, the pharmaceutical composition (or medicament) is administered to the subject through intralesional injection.

In one embodiment, the pharmaceutical composition (or medicament) comprises about 25% (w/v) sodium thiosulfate.

In one embodiment, the metabisulfite is sodium metabisulfite (SMB).

In one embodiment, the pharmaceutical composition (or medicament) comprises about 25% (w/v) sodium metabisulfite.

In one embodiment, the pharmaceutical composition (or medicament) is administered topically to the subject.

In one embodiment, the calcium phosphate tissue filler comprises calcium hydroxyapatite.

In one embodiment, the volume ratio of the tissue filler to the pharmaceutical composition ranges from about 4:1 to about 1:3.

In one embodiment, the volume ratio of the tissue filler to the pharmaceutical composition is about 1:1, 1:2 or 1:2.5.

### 5. BRIEF DESCRIPTION OF THE DRAWINGS

FIGS. 1A-1D. Hematoxylin and eosin stain (H&E stain) of a punch biopsy of the porcine samples (1) 24 hours with no treatment (control group: samples were not treated after the Radiesse® injection. FIG. 1A): or (2) 24 hours after treatment with: sodium thiosulfate (STS: FIG. 1B): sodium metabisulfite (SMB; FIG. 1C), or both STS and SMB (FIG. 1D). Note bright deposits of calcium in FIG. 1A.

FIGS. 1E-1F. Hematoxylin and eosin stain (H&E stain) of a punch biopsy of the human tissue samples 24 hours after treatment with: both STS and SMB (FIG. 1E); or STS only (FIG. 1F).

### 6. DETAILED DESCRIPTION

The present disclosure provides for methods and compositions for reducing the amount of a calcium phosphate tissue filler (e.g., calcium hydroxyapatite) implanted in a subject. In one embodiment, the pharmaceutical composition contains a thiosulfate (e.g., sodium thiosulfate), a metabisulfite (e.g., sodium metabisulfite), or a combination thereof. In one embodiment, the composition is administered to the subject through injection. In one embodiment, the composition is administered to the subject through intralesional injection. In certain embodiments, the composition is in the form of a gel, a cream, a lotion, etc. for topical application. In certain embodiments, the present composition is a solution (e.g., an aqueous solution).

In one embodiment, the subject in need of the presently disclosed methods or compositions has one or more tissue fillers implanted in a plastic and/or reconstructive surgery. In one embodiment, the tissue filler has been implanted for hard or soft tissue augmentation, regeneration, and/or repair. In one embodiment, a calcium phosphate tissue filler has been injected into a subject for hand augmentation. In certain embodiments, a calcium phosphate tissue filler has been used to correct or improve skin contour deficiencies, such as frown lines, worry lines, wrinkles, crow's feet, marionette lines, stretch marks, scars, nasolabial folds, or combinations thereof. In certain embodiments, a calcium phosphate tissue filler has been used to restore and/or correct the signs of facial fat loss (lipoatrophy) in people with human immunodeficiency virus. In certain embodiments, a calcium phosphate tissue filler has been injected to facial regions including, but not limited to, glabellar lines, subdermal support of the brows, malar and buccal fat pads, tear troughs, nasolabial folds, nose, lips, perioral region, marionette lines, oral commisures, chin, and/or saucerized acne scars. In certain embodiments, a calcium phosphate tissue filler has been used for nipple projection, nipple areolar reconstruction, treating urinary incontinence, treating vesicoureteral reflux, vocal cord augmentation, and as a radiographic tissue marker.

In certain embodiments, a subject is treated by the present compositions and methods due to complications caused by a tissue filler implanted in the subject. In certain embodiments, the present compositions and methods may be used to reduce the amount of a tissue filler.

In certain embodiments, the present composition is administered to the subject about 10 minutes, about 20 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 36 hours, about 2 days, about 3 days, about 4 days, about 5 days, about 6 days, about 1 week, about 2 weeks, about 4 weeks, about 1 month, about 2 months, about 3 months, about 4 months, about 5 months, about 6 months, about 7 months, about 8 months, about 9 months, about 10 months, about 11 months, about 1 year, about 1.5 years, about 2 years, about 2.5 years, about 3 years, about 3.5 years, about 4 years, about 4.5 years, about 5 years, or about more than 5 year, after administration (e.g., implantation) of the tissue filler.

In certain embodiments, the amount of a thiosulfate and/or a metabisulfite that is used in the present methods and compositions depends on the amount of the tissue filler that was implanted into a subject in need thereof. In certain embodiments, the volume ratio of the tissue filler to the present composition (e.g., comprising a thiosulfate and/or a metabisulfite) ranges from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 4:1 to about 1:3, from about 3:1 to about 1:3, from about 2:1 to about 1:3, from about 2:1 to about 1:2.5, from about 2:1 to about 1:2, from about 1:1 to about 1:3, from about 1:1 to about 1:2, from about 0.5:1 to about 0.6:1, from about 0.6:1 to about 0.8:1, from about 0.8:1 to about 1:1, from about 1:1 to about 1:1.5, from about 1:1 to about 1:2.5, from about 1:1.5 to about 1:2, from about 1:2 to about 1:2.5, from about 1:2.5 to about 1:3, from about 1:1 to about 2:1, from about 1:1 to about 3:1, from about 2:1 to about 3:1, from about 2:1 to about 4:1, about 4:1, about 2:1, about 1:1, to about 1:2, or about 1:2.5. In a specific embodiment, the tissue filler is calcium hydroxyapatite. In a specific embodiment, the thiosulfate is sodium thiosulfate.

Also encompassed by the present disclosure are methods and compositions for treating or preventing a calcification disorder, such as a cutaneous calcification (calcinosis cutis), a subcutaneous calcification, or a cardiovascular calcification. In one embodiment, the calcification disorder is a systemic calcium hydroxyapatite deposition disease. In certain embodiments, the pharmaceutical composition contains a thiosulfate (e.g., sodium thiosulfate), a metabisulfite (e.g., sodium metabisulfite), or a combination thereof. In certain embodiments, the present composition is administered to the subject through systemic administration (e.g., an intravenous route) or topical administration.

The present methods and compositions may be used to treat calciphylaxis which is a cutaneous or system disorder. The present methods and compositions may be used to reduce the amount of a calcium phosphate tissue filler (e.g., Radiesse®).

In one embodiment, the present composition comprises thiosulfate, metabisulfite or a combination thereof. The composition can be used in the presently disclosed methods.

The route of administration of the present composition can be chosen based on, for example, whether local or systemic treatment is desired, and on the area to be treated. For example, the compositions can be administered topically (including transdermally, subdermally, ophthalmically, vaginally, rectally, intranasally), orally, parenterally (e.g., intravenous, subcutaneous, intradermal, intraperitoneal, or intramuscular injection), by inhalation, extracorporeally, or the like. In some embodiments, the present composition is administered directly to the site of tissue filler implantation, or to the site of calcification using a drug delivery device or formulation. In one embodiment, the present composition may be injected directly into a lesion, a tissue, or an organ.

In certain embodiments, the present composition is in the form of a rapid release formulation, in the form of an extended release formulation, or in the form of an intermediate release formulation. In specific embodiments, sustained-release formulations may be administered by implantation (for example subcutaneously or intramuscularly), by a depot preparation, or by intramuscular or subcutaneous injection.

In certain embodiments, the present composition is used to reduce the amount of a tissue filler composition (e.g., implanted in a subject), calcium phosphate, or calcium salts.

In certain embodiments, the amount of a tissue filler composition, calcium phosphate, or calcium salts are reduced by, greater than (or at least) 50%, greater than (or at least) 60%, greater than (or at least) 70%, greater than (or at least) 80%, greater than (or at least) 90%, greater than (or at least) 95%, greater than (or at least) 97%, or greater than (or at least) 99%, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 36 hours, about 2 days, about 3 days, about 4 days, about 5 days, about 6 days, about 1 week, about 2 weeks, about 4 weeks, about 1 month, about 2 months, about 3 months, about 4 months, about 5 months, about 6 months, or within about 1 year, after administration of the present composition.

As used herein, the term “about” refers to ±10% of the referenced value.

In certain embodiments, the present compositions and methods are used to reduce the amount of tissue filler. In certain embodiments, the amount of the tissue filler is reduced at a localized area in the subject's body. In certain embodiments, the localized area in the subject's body has a size of about 1-5 mm², about 5-10 mm², about 10-15 mm², about 15-20 mm², about 20-25 mm², about 25-30 mm², about 30-40 mm², about 40-50 mm², about 50-60 mm², about 60-70 mm², about 70-80 mm², about 80-90 mm², about 90-100 mm², about 100-200 mm², about 200-300 mm², about 300-400 mm², about 400-500 mm², about 500-600 mm², about 600-700 mm², about 700-800 mm², about 800-900 mm², about 900-1000 mm², about 1-2 cm², about 2-3 cm², about 3-4 cm², about 4-5 cm², about 5-6 cm², about 6-7 cm², about 7-8 cm², about 8-9 cm², about 9-10 cm², about 10-11 cm², about 11-12 cm², or about 12-15 cm². In certain embodiments, the localized area in the subject's body has a size of about 1-5 mm³, about 5-10 mm³, about 10-15 mm³, about 15-20 mm³, about 20-25 mm³, about 25-30 mm³, about 30-40 mm³, about 40-50 mm³, about 50-60 mm³, about 60-70 mm³, about 70-80 mm³, about 80-90 mm, about 90-100 mm³, about 100-200 mm³, about 200-300 mm², about 300-400 mm³, about 400-500 mm³, about 500-600 mm³, about 600-700 mm³, about 700-800 mm³, about 800-900 mm³, about 900-1000 mm³, about 1-2 cm³, about 2-3 cm³, about 3-4 cm³, about 4-5 cm³, about 5-6 cm³, about 6-7 cm³, about 7-8 cm³, about 8-9 cm³, about 9-10 cm³, about 10-11 cm³, about 11-12 cm³, or about 12-15 cm³. The term “about” refers to ±10% of the referenced value. In one embodiment, the amount of the tissue filler is reduced by degradation. In one embodiment, the tissue filler is dissolved. In one embodiment, the tissue filler is converted into at least one or more soluble compounds. In one embodiment, the tissue filler is soluble. In one embodiment, after administration of the present composition, the tissue filler is removed from a localized area in the subject's body via the blood stream or interstitial fluid. In certain embodiments, the tissue filler is degraded by the present compositions and methods. In certain embodiments, the tissue filler composition is dissolved by the present compositions and methods. In one embodiment, after administration of the present composition, the tissue filler composition has increased solubility compared to that before administration of the present composition, and is carried away by the blood stream or interstitial fluid. In certain embodiments, the amount of the calcium phosphate tissue filler is reduced as the present compositions and methods convert calcium phosphate to more soluble calcium thiosulfate and/or calcium metabisulfite.

The term “about” in reference to a numeric value in the present disclosure refers to ±10% of the stated numeric value. In other words, the numeric value can be in a range of 90% of the stated value to 110% of the stated value.

In certain embodiments, the present compositions and methods are used to treat or prevent a calcification disorder. In one embodiment, a calcification disorder is caused by, or results from injection of a calcium phosphate tissue filler, such as a calcium phosphate tissue filler comprising calcium hydroxylapatite.

### 6.1 Thiosulfates

A thiosulfate (S₂O₃²⁻) is a sulfate ion with one oxygen replaced by sulfur. Non-limiting examples of thiosulfate salts include the alkali metal salts, alkaline earth metal salts or other types of salts, such as sodium, potassium, magnesium, lithium, aluminum salts and the like. Thiosulfates may be sodium thiosulfate, potassium thiosulfate, magnesium thiosulfate, lithium thiosulfate, or a combination thereof.

Sodium thiosulfate (STS) refers to any compound having the anhydrous formula Na₂S₂O₃ or any compound commonly known by the following names: hyposulphate of sodium, disodium salt of thiosulfuric acid, sodium hyposulphate, natrii thiosulfas, sodium thiosulphate, natrium thiosulfuricum, and natrium thiosulfate.

As used herein, sodium thiosulfate includes anhydrous, monohydrate, dihydrate, trihydrate, quatrahydrate, pentahydrate, and other hydrated forms of sodium thiosulfate or a combination thereof. In one embodiment, sodium thiosulfate refers to sodium thiosulfate pentahydrate (Na₂S₂O₃.5H₂O).

In one embodiment, thiosulfates of the present composition are pharmaceutical grade.

In certain embodiments, the content by weight of a thiosulfate in the present composition ranges from about 5% (w/v) to about 25% (w/v), from about 5% (w/v) to about 15% (w/v), from about 8% (w/v) to about 12% (w/v), from about 10% (w/v) to about 25% (w/v), from about 5% (w/v) to about 50% (w/v), from about 25% (w/v) to about 50% (w/v), from about 20% (w/v) to about 30% (w/v), from about 20% (w/v) to about 40% (w/v), from about 20% (w/v) to about 50% (w/v), from about 15% (w/v) to about 35% (w/v), from about 15% (w/v) to about 30% (w/v), from about 20% (w/v) to about 35% (w/v), from about 10% (w/v) to about 30% (w/v), from about 10% (w/v) to about 40% (w/v), from about 20% (w/v) to about 40% (w/v), from about 0.01% (w/v) to about 100% (w/v), from about 0.1% (w/v) to about 80% (w/v), from about 1% (w/v) to about 70% (w/v), from about 10% (w/v) to about 60% (w/v), from about 0.1% (w/v) to about 20% (w/v), about 10% (w/v), about 15% (w/v), about 20% (w/v), about 25% (w/v), about 30% (w/v), about 35% (w/v), about 40% (w/v), or about 45% (w/v), relative to the total volume of the composition.

In certain embodiments, the content by weight of a thiosulfate in the present composition ranges from about 5% (w/w) to about 25% (w/w), from about 5% (w/w) to about 15% (w/w), from about 8% (w/w) to about 12% (w/w), from about 10% (w/w) to about 25% (w/w), from about 5% (w/w) to about 50% (w/w), from about 25% (w/w) to about 50% (w/w), from about 20% (w/w) to about 30% (w/w), from about 20% (w/w) to about 40% (w/w), from about 20% (w/w) to about 50% (w/w), from about 0.01% (w/w) to about 100% (w/w), from about 0.1% (w/w) to about 80% (w/w), from about 1% (w/w) to about 70% (w/w), from about 10% (w/w) to about 60% (w/w), from about 0.1% (w/w) to about 20% (w/w), about 10% (w/w), about 12.5% (w/w), about 15% (w/w), about 20% (w/w), about 25% (w/w), about 30% (w/w), about 35% (w/w), about 40% (w/w), or about 45% (w/w), relative to the total weight of the composition.

In certain embodiments, the present composition is a solution (e.g., an aqueous solution) comprising, e.g., 10%-15% (w/v) of a thiosulfate, 15%-20% (w/v) of a thiosulfate, 20%-25% (w/v) of a thiosulfate, 25%-30% (w/v) of a thiosulfate, or any other concentrations of a thiosulfate as described herein.

In certain embodiments, the volume of the present composition for injection per injection site is 0.1 ml-0.2 ml, 0.2 ml-0.4 ml, 0.4 ml-0.5 ml, 0.4 ml-0.8 ml, 0.5 ml-0.8 ml or 0.8 ml-1 ml.

In a specific embodiment, 0.2 ml of 25% w/v sodium thiosulfate may be used to effectively dissolve 0.4 ml-0.8 ml of Calcium hydroxylapatite tissue filler in a localized area in a subject.

### 6.2 Metabisulfite

A metabisulfite, also known as disulfite or pyrosulfite, is a chemical compound containing the metabisulfite ion (disulfite ion) S₂O₅²⁻. Non-limiting examples of metabisulfite salts include the alkali metal salts, alkaline earth metal salts or other types of salts, such as sodium, potassium, magnesium, lithium, aluminum salts or a combination thereof. In certain embodiments, metabisulfite is sodium metabisulfite, potassium metabisulfite, magnesium metabisulfite, lithium metabisulfite, or a combination thereof.

Sodium metabisulfite (SMB) refers to any compound having the anhydrous formula Na₂S₂O₅ or any compound commonly known by the following names: Sodium pyrosulfite, and

Sodium disulfite.

In one embodiment, metabisulfites of the present composition are pharmaceutical grade.

In certain embodiments, the content by weight of a metabisulfite in the present composition ranges from about 5% (w/v) to about 25% (w/v), from about 5% (w/v) to about 15% (w/v), from about 8% (w/v) to about 12% (w/v), from about 10% (w/v) to about 25% (w/v), from about 5% (w/v) to about 50% (w/v), from about 25% (w/v) to about 50% (w/v), from about 20% (w/v) to about 30% (w/v), from about 20% (w/v) to about 40% (w/v), from about 20% (w/v) to about 50% (w/v), from about 15% (w/v) to about 35% (w/v), from about 15% (w/v) to about 30% (w/v), from about 20% (w/v) to about 35% (w/v), from about 10% (w/v) to about 30% (w/v), from about 10% (w/v) to about 40% (w/v), from about 20% (w/v) to about 40% (w/v), from about 0.01% (w/v) to about 100% (w/v), from about 0.1% (w/v) to about 80% (w/v), from about 1% (w/v) to about 70% (w/v), from about 10% (w/v) to about 60% (w/v), from about 0.1% (w/v) to about 20% (w/v), about 10% (w/v), about 15% (w/v), about 20% (w/v), about 25% (w/v), about 30% (w/v), about 35% (w/v), about 40% (w/v), or about 45% (w/v), relative to the total volume of the composition.

In certain embodiments, the content by weight of a metabisulfite in the present composition ranges from about 5% (w/w) to about 25% (w/w), from about 5% (w/w) to about 15% (w/w), from about 8% (w/w) to about 12% (w/w), from about 10% (w/w) to about 25% (w/w), from about 5% (w/w) to about 50% (w/w), from about 25% (w/w) to about 50% (w/w), from about 20% (w/w) to about 30% (w/w), from about 20% (w/w) to about 40% (w/w), from about 20% (w/w) to about 50% (w/w), from about 0.01% (w/w) to about 100% (w/w), from about 0.1% (w/w) to about 80% (w/w), from about 1% (w/w) to about 70% (w/w), from about 10% (w/w) to about 60% (w/w), from about 0.1% (w/w) to about 20% (w/w), about 10% (w/w), about 15% (w/w), about 20% (w/w), about 25% (w/w), about 30% (w/w), about 35% (w/w), about 40% (w/w), or about 45% (w/w), relative to the total weight of the composition.

In certain embodiments, the present composition comprises a metabisulfite and a thickener or gelling agent, including, but not limited to, petrolatum, petroleum, a paraffin-type wax, cellulose and derivatives thereof, polysaccharides, carbomers, polyvinyl alcohol, povidone, colloidal silicon dioxide, cetyl alcohols, stearic acid, beeswax, petrolatum, triglycerides, lanolin, and combinations thereof. In certain embodiments, the present composition comprises a metabisulfite and petrolatum.

In certain embodiments, the volume of the present composition for topical application site is 0.1 ml-0.2 ml, 0.2 ml-0.4 ml, 0.4 ml-0.5 ml, 0.4 ml-0.8 ml, 0.5 ml-0.8 ml or 0.8 ml-1 ml.

### 6.3 Tissue Filler

The tissue filler composition disclosed herein is used in soft tissue and/or hard tissue repair and/or augmentation. In certain embodiments, the tissue filler composition is used in soft tissue repair, hard bone replacement, bone regeneration, for filling solid tissue voids, treatment of skin contour deficiencies, and/or for cosmetic use in plastic surgery or in reconstructive surgery. In certain embodiments, the tissue filler composition is used for regeneration of tissues, such as bone, cartilage, connective tissues and the like. In certain embodiments, the tissue filler composition may be formed into implants. In certain embodiments, the tissue filler composition is used to treat skin deficiencies caused by diseases such as acne, cancer and lipodystrophy syndrome. In certain embodiments, the tissue filler composition is used to treat scars on or within the skin. In certain embodiments, the skin deficiencies are the result of a disease, the treatment of a disease, caused by accidents, wounds or injuries. In certain embodiments, the tissue filler composition is used for the treatment of skin contour deficiencies, which may be caused by aging, environmental exposure, weight loss, child bearing, injury and surgery. Non-limiting examples of skin contour deficiencies include frown lines, worry lines, wrinkles, crow's feet, marionette lines, stretch marks, and internal and external scars (e.g., resulted from injury, wound, surgery, or accident). In certain embodiments, the skin contour deficiencies treated by the tissue filler composition is in areas such as face, cheeks, lip, eye lid, nose, forehead, neck, arm, shoulder, buttock, and thigh.

In certain embodiments, the tissue filler composition may be applied to augment and/or repair internal tissues, such as tissue defining sphincters in the treatment of incontinence, and for the treatment of unilateral vocal cord paralysis.

In one embodiment, the tissue filler composition is delivered to the target site by injection.

In certain embodiments, the tissue filler composition comprises calcium phosphate, including, but not limited to, calcium hydroxyapatite (CaHA, also known as basic calcium phosphate), amorphous calcium phosphates (ACP), tetracalcium phosphate (TTCP), calcium pyrophosphate, monocalcium phosphates (MCPM), dicalcium phosphates (DCPA), tricalcium phosphate (TCP), octacalcium phosphate, calcium fluorapatite, calcium carbonate apatite, or combinations thereof. In certain embodiments, the tissue filler composition comprises hydroxyapatite derivatives. In certain embodiments, the tissue filler composition comprises calcium silicate, calcium carbonate, fluorapatite, fluorides or combinations thereof.

In certain embodiments, the tissue filler composition comprises calcium hydroxyapatite and a second tissue filler. In certain embodiments, the tissue filler composition comprises calcium hydroxyapatite and hyaluronic acid. In certain embodiments, weight ratio (or volume ratio, or molar ratio) of calcium hydroxyapatite and hyaluronic acid ranges from about 0.5:1 to about 0.6:1, from about 0.6:1 to about 0.8:1, from about 0.8:1 to about 1:1, from about 1:1 to about 1:1.5, from about 1:1.5 to about 1:2, from about 1:2 to about 1:2.5, or from about 1:2.5 to about 1:3.

In certain embodiments, the tissue filler composition comprises calcium hydroxyapatite and one or more other tissue filler. In certain embodiments, the tissue filler composition comprises calcium hydroxyapatite and one or more pharmaceutically acceptable carriers as described herein.

Hydroxyapatite, also known as hydroxylapatite, comprises calcium, phosphate and hydroxyl groups and has the chemical formula Ca₅(PO₄)₃(OH). Hydroxyapatite is commonly written as Ca₁₀(PO₄)₆(OH)₂ to denote that the crystal unit comprises two hydroxyapatite molecules.

In one embodiment, the tissue filler is the injectable dermal filler Radiesse® (Merz North America, Inc.). Radiesse® is an opaque dermal filler composed of synthetic calcium hydroxylapatite (CaHA) microspheres (particles) suspended in a water-based gel carrier. In one embodiment, Radiesse® comprises synthetic calcium hydroxylapatite, water, glycerin and sodium carboxymethylcellulose. In certain embodiments, the sizes of the CaHA particles range from about 5 microns to about 100 microns, from about 10 microns to about 80 microns, from about 20 microns to about 60 microns, from about 10 microns to about 50 microns, from about 25 microns to about 45 microns, or from about 25 microns to about 40 microns. In certain embodiments, the sizes of the CaHA particles range from about 25 microns to about 45 microns.

In one embodiment, the tissue filler is Radiesse® (+) (Merz North America. Inc.). Radiesse® (+) contains a small quantity (e.g., 0.3%) of local anesthetic (e.g., lidocaine), shown to reduce pain related to the injection.

In certain embodiments, the volume of the tissue filler for administration (e.g., injection) per administration site (e.g., per injection site) is 0, ml-0.2 ml, 0.2 ml-0.4 ml, 0.4 ml-0.5 ml, 0.5 ml-0.8 ml or 0.8 ml-1 ml.

In certain embodiments, the present compositions and methods are used to reduce the amount of a tissue filler, and/or treat a calcification disorder caused by and/or resulting from injection of a calcium phosphate tissue filler. In certain embodiments, a subject is treated by the present compositions and methods due to complications caused by a tissue filler implanted in the subject. In certain embodiments, the complications may be nodules, residual palpable or visible material, an uneven surface, blanching, hypersensitivity reactions or a combination thereof, following tissue filler administration. In certain embodiments, the complications may be vascular compromise (such as arterial embolization, venous obstruction, etc.) caused, e.g., by excessive injection volume. In certain embodiments, the complications may include pain, edema, excessive swelling, geographic discoloration, excessive bruising, ecchymosis, excessive tenderness, worsening of patient appearance, migration of the filler, and/or granuloma formation. In certain embodiments, the complications may be aesthetically displeasing. In one embodiment, if the injected material is placed too superficially or in excess quantity, it can result in visible bumps or blanching/bluish discoloration of the surrounding area. In certain embodiments, major complications may produce permanent, disfiguring damage. Liao et al., Soft Tissue Fillers: Avoiding and Treating complications. EyeNet Magazine, February 2013.

In certain embodiments, the complications are early-onset complications that occur within the first two weeks after the tissue filler administration, or delayed-onset complications that occur after the first two weeks post-tissue filler administration.

### 6.4 Calcification Disorders

The present compositions disclosed herein are used to treat and/or prevent calcification disorders. In certain embodiments, the disclosure provides a method of decreasing or preventing pathological calcification in an individual. In some embodiments, administration of a therapeutically effective amount of the present composition retards or reverses the formation, growth or deposition of calcium salts (e.g., extracellular matrix hydroxyapatite deposits).

In some embodiments, a subject treated with the methods and compositions provided herein is a human who has or is diagnosed with a calcification disorder. In other embodiments, a subject treated with the methods and compositions provided herein is a human predisposed or susceptible to a calcification disorder. In some embodiments, a subject treated with the methods and compositions provided herein is a human at risk of developing a calcification disorder. In other embodiments, a subject treated with the methods and compositions provided herein is a human who has developed, or is developing, a calcification disorder from injection of a calcium phosphate tissue filler.

A calcification disorder (or a pathological calcification) refers to inappropriate biomineralization (pathological deposit of calcium salts or any bone growth) in soft tissues (Giachelli (1999) American Journal of Pathology, 154, 671-675). A calcification disorder may refer to any formation, growth or deposition of extracellular matrix hydroxyapatite (calcium phosphate) deposits in any tissue other than bone, growth-plate cartilage and dentin, or to calcification in bone, growth-plate cartilage and dentin that is not part of normal development and maintenance of the skeletal system.

The term “soft tissue” refers to a tissue other than bone tissue, which connects, supports, or surrounds other structures and organs of the body. If the injected material is placed too superficially or in excess quantity, it can result in visible bumps or blanching/bluish discoloration of the surrounding area. In certain embodiments, a calcification disorder occurs in soft tissues, including, but not limited to, skin, tendons, ligaments, cartilage, fascia, fibrous tissues, adipose tissues, synovial membranes, muscles, nerves, blood vessels, cardiovascular tissues, etc. In certain embodiments, a calcification disorder occurs in breasts, ovaries, uterus, kidney, heart valves, as well as in or around a prosthetic device. Kirsch, Curr. Opin. Rhematol. 2006: 18: 174-180.

In certain embodiments, calcifications comprise calcium phosphate salts (e.g., hydroxyapatite), calcium oxalates, and/or octacalcium phosphate or a combination thereof.

In certain embodiments, calcification disorders are dystrophic, metastatic, idiopathic, or iatrogenic calcifications, subcutaneous ectopic ossifications, or calciphylaxis. In certain embodiments, calcification disorders are skeletal calcification, or extraskeletal calcification.

Dystrophic calcifications are the result of local tissue abnormalities and grow in spite of normal plasma calcium and phosphorus levels. In certain embodiments, the main diseases that may develop due to these calcifications are: connective tissue diseases (e.g., scleroderma, CREST syndrome, juvenile dermatomyositis, lupus), cutaneous and subcutaneous infections (e.g., panniculitis), skin tumors (e.g., pilomatricoma), certain congenital diseases (e.g., Elher-Danlos disease, Werner's syndrome, pseudo xanthoma elasticum).

Metastatic calcifications are the result of a disorder of calcium and phosphate metabolism (hypercalcemia and/or hyperphosphatemia). All diseases that cause these disorders may therefore contribute to the development of calcifications.

Idiopathic calcifications occur without tissue lesions or disorders of calcium and phosphate metabolism. The main known diseases in this group are tumoral calcinosis, scrotal calcifications as well as sub-epidermal calcified nodules.

Iatrogenic calcifications can occur following the injection of calcium or para-aminosalycylic acid. They have also been described following the use of calcium chloride saturated electrodes.

Calciphylaxis corresponds to the calcification of small sized blood vessels and of the sub-cutaneous adipose tissue. Most of the time secondary to chronic renal failure, and often associated with abnormalities of calcium and phosphate metabolism, calciphylaxis exhibits specific pathophysiology and particular evolutionary modalities.

In certain embodiments, calcifications that may be treated using the methods and compositions of the present disclosure are located in the cutaneous and subcutaneous tissues. Calcification disorders treated by the present compositions may be cutaneous or subcutaneous calcifications.

Calcification disorders may include, but are not limited to, scleroderma, urethral stone, cardiac valve mineralization, atherosclerosis, ankylosing spondylitis, tumoral calcinosis, fibrodysplasia ossificans progressiva, progressive osseous heteroplasia, pseudoxanthoma elasticum, ankylosis, osteoarthritis, general arterial calcification in infancy (GACI), arterial calcification due to deficiency of CD73 (ACDC), Keutel syndrome, peritoneal calcification, heterotopic calcification in amputees, tibial artery calcification, bone metastasis, prosthetic calcification, or Paget's disease of bone.

The present compositions and methods may be used to treat calcifications associated with a disease or pathological condition including, but not limited to, primary hyperparathyroidism, vitamin D intoxication, milk drinker's syndrome, hypercalcemia, secondary hyperparathyroidism, renal failure, hyperphosphatemia, in particular genetic hyperphosphatemia, scleroderma, dermatomyositis, in particular the juvenile form, mixed connective tissue diseases, lupus, CREST syndrome. Elhers-Danlos syndrome, pseudo xanthoma elasticum, Werner's syndrome, late cutaneous porphyria, pseudo hypoparathyroidism, pseudo pseudo-hypoparathyroidism, (primary or secondary) venous or arterial insufficiency, diabetes, scrotal calcinosis, ossifying myositis, post-traumatic ectopic ossifications and any other disease or pathological condition caused by calcium crystal deposit(s), in particular of hydroxyapatite or calcium pyrophosphate.

Calcification may be caused by a valvular disease, hyperlipidaemia, senility, estrogen deficiency, angina, heart failure, nephritis, uremia, diabetes, an inflammatory disease, or a cardiovascular disorder. Examples of the nephritis may include glomerulonephritis, diabetic nephritis, lupus nephritis, multiple pustular nephritis, pyelonephritis, lithonephria, nephrotuberculosis, renal tumor, and the like. Examples of the inflammatory disease may include asthma, allergic and non-allergic rhinitis, chronic and acute rhinitis, chronic and acute gastritis or enteritis, ulcerative gastritis, acute and chronic nephritis, acute and chronic hepatitis, a chronic obstructive pulmonary disease, pulmonary fibrosis, an irritable bowel syndrome, inflammatory pain, migraine headache, headache, backache, a fibromyalgia syndrome, a myofascial disease, a viral infection (for example, hepatitis C), a bacterial infection, a fungal infection, a burn, an injury caused by a surgical or dental operation, a prostaglandin E overload syndrome, atherosclerosis, gout, arthritis, rheumatic arthritis, ankylosing spondylitis, a Hodgkin's disease, pancreatitis, conjunctivitis, iritis, scleratitis, uveitis, dermatitis, eczema, or multiple sclerosis. In addition, examples of the cardiovascular disease may include myocardiopathy, a primary cardiac arrest, ischemic heart failure, hypertension, an ischemic heart disease, a coronary artery disease, angina, myocardial infarction, atherosclerosis, or arrhythmia.

The present compositions and methods may be used to treat complications related to a calcification disorder. This type of complication may, for example, be a functional complication (e.g., a limitation of range of motion and joint function), pain, atrophic complication (e.g., ischemia or necrosis of the cutaneous and/or subcutaneous tissues), or an infection.

Calcification disorders may include vascular or valvular calcification which refers to formation, growth and deposition of extracellular matrix hydroxyapatite (calcium phosphate) crystal deposits in the blood vessels or valves. Vascular calcification encompasses coronary, valvular, aortic, and other blood vessel calcification. Vascular calcification includes atherosclerotic and medial wall calcification. Both forms of vascular calcification are associated with various diseases and disorders. For instance, both atherosclerotic and medial calcification has been found to be common in uremic patients (Proudfoot, D & Shanahan, C. Herz. 26: 245-51, 2001; Chen et al., Semin. Nephrol. 24: 61-8, 2004) and in patients with diabetes mellitus I and II. Conditions characterized by medial wall calcification include idiopathic infantile arterial calcification (IIAC), Kawasaki disease, end-stage renal disease, diabetes, and obesity. Medial wall calcification is also a general characteristic of increased age. Vascular calcification is a common complication of chronic kidney disease (CKD) (Giachelli, C. J. Am. Soc. Nephrol. 15: 2959-64, 2004; Raggi, P. et al. J. Am. Coll. Cardiol. 39: 695-701, 2002).

### 6.5 Assessment of Calcification or Amount of Tissue Filler

Methods of detecting and measuring calcification are well known in the art. In some embodiments, methods of measuring calcification include direct methods of detecting and measuring extent of calcium-phosphorus depositions.

In some embodiments, methods of measuring/assessing calcification, or methods of measuring/assessing the amount of a calcium phosphate tissue filler, include, but are not limited to, in vivo imaging methods such as plain film roentgenography, coronary arteriography; fluoroscopy, including digital subtraction fluoroscopy; cinefluorography; computed tomography (CT); X-ray; helical computed tomography; spiral computed tomography; electron beam computed tomography (EBCT); intravascular ultrasound (IVUS); magnetic resonance imaging; and transthoracic and transesophageal echocardiography. Coronary interventionalists may use cinefluorography and IVUS to evaluate calcification in specific lesions before angioplasty. In some embodiments, CT can be done using double-helix CT scanners or single-helix scanners.

In some embodiments, calcification, or the amount of a calcium phosphate tissue filler, can be measured by magnetic resonance imaging (MRI). In some embodiments, calcification, or the amount of a calcium phosphate tissue filler, can be measured by transthoracic (surface) echocardiography. In some embodiments, calcification, or the amount of a calcium phosphate tissue filler, can be assessed ex vivo by Van Kossa method. This method relies upon the principle that silver ions can be displaced from solution by carbonate or phosphate ions due to their respective positions in the electrochemical series. The argentaffin reaction is photochemical in nature and the activation energy is supplied from strong visible or ultra-violet light. Since the demonstrable forms of tissue carbonate or phosphate ions are invariably associated with calcium ions the method can be considered as demonstrating sites of tissue calcium deposition.

Other methods of measuring calcification, or the amount of a calcium phosphate tissue filler, include, but are not limited to, immuno-fluorescent staining and densitometry. In another aspect, methods of assessing calcification, or the amount of a calcium phosphate tissue filler, include methods of measuring determinants and/or risk factors of vascular calcification. Such factors include, but are not limited to, serum levels of phosphorus, calcium, and calcium phosphorus product, parathymid hormone (PTH), low-density lipoprotein cholesterol (LDL), high-density lipoprotein cholesterol (HDL), triglycerides, and creatinine. Methods of measuring these factors are well known in the art. Other methods of assessing calcification, or the amount of a calcium phosphate tissue filler, include assessing factors of bone formation. Such factors include bone formation markers such as bone-specific alkaline phosphatase (BSAP), osteocalcin (OC), carboxyterminal propeptide of type I collagen (PICP), and aminoterminal propeptide of type I collagen (PINP); serum bone resorption markers such as cross-linked C-telopeptide of type I collagen (ICTP), tartrate-resistant acid phosphatase, TRACP and TRAPSB, N-telopeptide of collagen cross-links (NTx), and C-telopeptide of collagen cross-links (CTx); and urine bone resorption markers, such as hydroxyproline, free and total pyridinolines (Pyd), free and total deoxypyridinolines (Dpd), N-telopeptide of collagen cross-links (NTx), and C-telopeptide of collagen crosslinks (CTx).

In certain embodiments, the calcium hydroxylapatite (CaHA) particles of Radiesse® injectable implant are radiopaque and are visible on CT Scans and/or in standard, plain radiography.

In certain embodiments, Radiesse® injectable implant contains calcium hydroxylapatite particles that are radiopaque and suspended in a water based gel. The radiographic assessment consisted of standard, plain radiography and/or CT scanning. X-rays and/or CT Scans were assessed by two or more blinded, licensed radiologists.

### 6.6 Combination Therapy

The present pharmaceutical composition may be used alone, or may be used in combination with other treatments/agents.

When the present composition is used to reduce the amount of a tissue filler, the second agent/treatment used in combination with the present composition may include, but are not limited to, aspiration, injection, intralesional steroid injection, oral steroids, massage, dermabrasion, puncture and expression, oral allopurinol, injectable hyaluronidase, or surgical excision.

When the present composition is used to treat a calcification disorder, the second agent/treatment used in combination with the present composition may include, but are not limited to, surgery, hormone treatments, drug treatments, etc. Agents that may be used in combination with the present composition include, but are not limited to, bisphosphonates, calcium channel blockers, probenecid, vitamin D sterols, and/or Renagel®. Vitamin D sterols can include calcitriol, alfacalcidol, doxercalciferol, maxacalcitol or paricalcitol. In certain embodiments, the present composition is used with calcimimetics, vitamins and their analogs, antibiotics, lanthanum carbonate, lipid-lowering agents, anti-hypertensives, anti-inflammatory agents (steroidal and non-steroidal), inhibitors of pro-inflammatory cytokine, and cardiovascular agents.

The present composition may also contain, or be used in combination with, agents that provide therapeutic effects to the procedure or biological or physiological responses to the procedure. An example of such agents is an anti-inflammation agent, an anti-bacterial, anti-fungal or anti-histamine agent. Another suitable agent is a local anesthetic agent.

In certain embodiments, the subject may or may not be receiving any other preventive or therapeutic, pharmacological or mechanical medication for calcifications or for reducing the amount of tissue filler, in addition to the present pharmaceutical composition.

In certain embodiments, when the present composition is combined with one or more other agents, they may be mixed in a single pharmaceutical composition, or may be formulated into separate pharmaceutical compositions. In certain embodiments, the second agent can be formulated or packaged with the compound provided herein, according to those of skill in the art, such co-formulation should not interfere with the activity of either agent or the method of administration. In certain embodiments, the compound provided herein and the second agent are formulated separately. In one embodiment, they are packaged together.

In one embodiment, they are packaged separately.

When administered separately, the present composition may be administered simultaneously with, prior to, or after the other agents/treatments. The present composition may be administered within about 10 mins, 15 mins, 30 mins, 1 hour, within about 2 hours, within about 3 hours, within about 4 hours, within about 5 hours, within about 10 hours, within about 12 hours, within about 24 hours, within about 2 days, within about 3 days, within about 4 days, within about 5 days, within about 6 days, within about 1 week, within about 2 weeks, within about 3 weeks, within about 4 weeks, within about 1 month, within about 2 months, within about 3 months, within about 6 months, or within about 1 year, of administering the other therapy method(s).

In one embodiment, the method described herein comprises the use of a topical composition in conjunction with STS for added benefit with synergistic and more effective results. In one embodiment, the method described is used for the treatment of a calcification disorder in a subject comprising the step of administering to the subject an effective amount of a pharmaceutical composition comprising a thiosulfate, a metabisulfite, or a combination thereof. In certain embodiments, the method comprises an injection of the compositions described herein and a topical application of the compositions described herein. In certain embodiments, the method comprises a topical application of the compositions described herein and an injection of the compositions described herein.

Provided herein is a medical device (such as a catheter, a stent, etc.) coated with, or otherwise comprising, the compounds (or compositions) described herein. In certain embodiments, the medical device is an implantable medical device. In certain embodiments, the medical device is a surgical instrument. In one embodiment, the medical device is bioabsorbable.

In certain embodiments, the present composition may be administered in conjunction with prostheses, such as a prosthetic heart valve.

In certain embodiments, the present composition may be administered in combination with other therapies, such as epilation, UV irradiation, laser peeling, mechanical peeling, chemical peeling procedures, and combinations thereof.

### 6.7 Pharmaceutical Compositions

The compound disclosed herein can be formulated into pharmaceutical compositions using methods available in the art and those disclosed herein.

The methods provided herein encompass administering pharmaceutical compositions containing at least one compound as described herein, if appropriate in the salt form, either used alone or in the form of a combination with one or more compatible and pharmaceutically acceptable carriers, such as diluents or adjuvants.

In certain embodiments, the active agents provided herein may be administered by any conventional route, in particular topically, orally, parenterally, rectally or by inhalation (e.g., in the form of aerosols).

Topical administration refers to any administration by the local route, for example over the skin, an orifice, or a mucous membrane. Topical administration as used herein, includes the cutaneous, aural, nasal, vaginal, urethral, and rectal mutes of administration.

In certain embodiments, the compound provided herein is administered orally. In certain embodiments, the compounds may be made, as solid compositions for oral administration, of tablets, pills, hard gelatin capsules, powders or granules. In these compositions, the active product is mixed with one or more inert diluents or adjuvants, such as sucrose, lactose or starch. These compositions can comprise substances other than diluents, for example a lubricant, such as magnesium stearate, or a coating intended for controlled release. Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions for parenteral administration can be emulsions or sterile solutions. Use may be made, as solvent or vehicle, of propylene glycol, a polyethylene glycol, vegetable oils, or injectable organic esters, for example ethyl oleate. These compositions can also contain adjuvants, in particular wetting, isotonizing, emulsifying, dispersing and stabilizing agents.

Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In one embodiment, a composition provided herein is a pharmaceutical composition or a single unit dosage form. Pharmaceutical compositions and single unit dosage forms provided herein comprise a therapeutically effective amount of one or more therapeutic agents (e.g., a compound provided herein, or other prophylactic or therapeutic agent), and one or more pharmaceutically acceptable carriers or excipients.

The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin. Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form.

In certain embodiments, the composition comprises glycerin and/or sodium carboxymethylcellulose.

The composition or single unit dosage form, if desired, can also contain minor amounts of wetting or emulsifying agents, or pH buffering agents. Lactose free compositions provided herein can comprise excipients that are well known in the art and are listed, for example, in the U.S. Pharmocopia. In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre-gelatinized starch, and magnesium stearate. Further encompassed herein are anhydrous pharmaceutical compositions and dosage forms comprising active ingredients, since water can facilitate the degradation of some compounds. Anhydrous pharmaceutical compositions and dosage forms provided herein can be prepared using anhydrous or low moisture containing ingredients and low moisture or low humidity conditions. An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in certain embodiments, in purified form, together with a suitable amount of carrier so as to provide the form for proper administration to the subject. The formulation should suit the mode of administration. In a certain embodiment, the pharmaceutical compositions or single unit dosage forms are sterile and in suitable form for administration to a subject, for example, an animal subject, such as a mammalian subject, for example, a human subject.

A pharmaceutical composition is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer.

Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. For example, a dosage form used in the initial treatment of a condition may contain larger amounts of one or more of the active ingredients it comprises than a dosage form used in the maintenance treatment of the same condition. Similarly, a parenteral dosage form may contain smaller amounts of one or more of the active ingredients it comprises than an oral dosage form used to treat the same disease or disorder. These and other ways in which specific dosage forms encompassed herein will vary from one another will be readily apparent to those skilled in the art. See, e.g., Remington's Pharmaceutical Sciences, 20th ed., Mack Publishing. Easton Pa. (2000).

Typical dosage forms comprise a compound provided herein, or a pharmaceutically acceptable salt, solvate or hydrate thereof lie within the range of from about 0.1 mg to about 1000 mg per day, given as a single once-a-day dose in the morning or as divided doses throughout the day taken with food. Particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound. The term “about” refers to ±10% of the referenced value. Oral Dosage Forms Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, provided herein is a lotion comprising the compounds as disclosed herein.

### 6.8 Dosage and Unit Dosage Forms

A physician may determine the appropriate posology according to a preventive or curative treatment and according to the age, weight, stage of the disorder/condition and other factors specific to the subject to be treated.

In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated. The term “about” refers to ±10% of the referenced value.

Encompassed by the present disclosure are methods of degrading, or reducing the amount of a tissue filler. The amount of the composition which will be effective in degrading, dissolving or reducing the amount of a tissue filler will vary with the nature and severity of the condition caused by the tissue filler and the route by which the active ingredient is administered. The frequency and dosage will also vary according to factors specific for each subject depending on the specific therapy (e.g., therapeutic or prophylactic agents) administered, the severity of the condition/disorder, the route of administration, as well as age, body, weight, response, and the past medical history of the subject.

In further aspects, provided are methods of treating or preventing a calcification disorder in a subject by administering, to a subject in need thereof, an effective amount of a composition provided herein. The amount of the composition which will be effective in the prevention or treatment of a disorder or one or more symptoms thereof will vary with the nature and severity of the disorder, and the route by which the active ingredient is administered. The frequency and dosage will also vary according to factors specific for each subject depending on the specific therapy (e.g., therapeutic or prophylactic agents) administered, the severity of the condition/disorder, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a subject is 0.140 mg/kg to 3 mg/kg of the subject's body weight, based on weight of the active compound. In certain embodiments, the dosage administered to a subject is between 0.20 mg/kg and 2.00 mg/kg, or between 0.30 mg/kg and 1.50 mg/kg of the subject's body weight. The term “about” refers to ±10% of the referenced value.

In certain embodiments, the recommended daily dose range of a composition provided herein for the conditions described herein lie within the range of from about 0.1 mg to about 1000 mg per day, given as a single once-a-day dose or as divided doses throughout a day. In one embodiment, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response. The term “about” refers to ±10% of the referenced value. The term “about” refers to ±10% of the referenced value.

Different therapeutically effective amounts may be applicable for different conditions. Similarly, amounts sufficient to prevent, manage, treat or ameliorate such conditions, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need to be the same. For example, the dosage administered to the subject may be increased to improve the prophylactic or therapeutic effect of the composition or it may be decreased to reduce one or more side effects that a particular subject is experiencing.

In certain embodiment, the dosage of the composition provided herein, based on weight of the active compound, administered to prevent, treat, manage, or ameliorate a condition, or one or more symptoms thereof, in a subject is, 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a condition, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg. In certain embodiments, treatment or prevention can be initiated with one or more loading doses of a compound or composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. In certain embodiments, each maintenance does is, independently, about from about 10 mg to about 200 mg per day, between about 25 mg and about 150 mg per day, or between about 25 and about 80 mg per day. Maintenance doses can be administered daily and can be administered as single doses, or as divided doses. In certain embodiments, a dose of a compound or composition provided herein can be administered to achieve a steady-state concentration of the active ingredient in blood or serum of the subject. The steady-state concentration can be determined by measurement according to techniques available to those of skill or can be based on the physical characteristics of the subject such as height, weight and age. In certain embodiments, a sufficient amount of a compound or composition provided herein is administered to achieve a steady-state concentration in blood or serum of the subject of from about 300 to about 4000 ng/mL, from about 400 to about 1600 ng/mL, or from about 600 to about 1200 ng/mL. In some embodiments, loading doses can be administered to achieve steady-state blood or serum concentrations of about 1200 to about 8000 ng/mL, or about 2000 to about 4000 ng/mL for one to five days. In certain embodiments, maintenance doses can be administered to achieve a steady-state concentration in blood or serum of the subject of from about 300 to about 4000 ng/mL, from about 400 to about 1600 ng/mL, or from about 600 to about 1200 ng/mL. In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months. In certain aspects, provided herein are unit dosages comprising a compound, or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art. The term “about” refers to ±10% of the referenced value.

In various embodiments, the therapies (e.g., a composition provided herein and a second agent in a combination therapy) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the composition provided herein and the second agent are administered concurrently. In other embodiments, the composition provided herein and the second agent are administered at about 2 to 4 days apart, at about 4 to 6 days apart, at about 1 week part, at about 1 to 2 weeks apart, or more than 2 weeks apart.

In certain embodiments, administration of the same agent (e.g., the present composition) may be repeated and the administrations may be separated by at least 1 day, 2 days, 3 days, 4 days, 5 days, 6 days, a week, 8 days, 9 days, 10 days, 15 days, 30 days, 45 days, 2 months, 75 days, 3 months, 6 months, a longer time period, or as needed. In certain embodiments, administration of the same agent (e.g., the present composition) may be repeated and the administrations may be during one visit, 2, 3, 4, 5, 6, 7, 8, 9, 10 or more visits of the patient to the physician's office.

In certain embodiments, a composition provided herein and a second agent are administered to a patient, for example, a mammal, such as a human, in a sequence and within a time interval such that the composition provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the composition provided herein and the second active agent exerts their effect at times which overlap. Each second active agent can be administered separately, in any appropriate form and by any suitable route. In other embodiments, the composition provided herein is administered before, concurrently or after administration of the second active agent. The term “about” refers to ±10% of the referenced value. In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day. The second agent can act additively or synergistically with the compound provided herein. In one embodiment, the composition provided herein is administered concurrently with one or more second agents in the same pharmaceutical composition. In another embodiment, a composition provided herein is administered concurrently with one or more second agents in separate pharmaceutical compositions. In still another embodiment, a composition provided herein is administered prior to or subsequent to administration of a second agent. Also contemplated are administration of a composition provided herein and a second agent by the same or different routes of administration, e.g., oral and parenteral. In certain embodiments, when the composition provided herein is administered concurrently with a second agent that potentially produces adverse side effects including, but not limited to, toxicity, the second active agent can advantageously be administered at a dose that falls below the threshold that the adverse side effect is elicited.

### 6.9 Kits

Also provided are kits for use in methods of degrading, dissolving, and/or reducing the amount of a tissue filler. The present disclosure provides kits for use in methods for treating or preventing a calcification disorder.

The kits can include a compound or composition provided herein, and instructions providing information to a health care provider regarding usage in accordance with the present methods. The kit may optionally contain a second agent or composition.

The present disclosure provides for a pharmaceutical combination, preferably in a kit, comprising: (a) a calcium phosphate tissue filler; and (b) a pharmaceutical composition comprising a thiosulfate, a metabisulfite, or combination thereof.

In certain embodiment, the pharmaceutical combination comprises about 25% (w/v) of the thiosulfate, metabisulfite, or combination thereof, or any other concentrations of the thiosulfate, metabisulfite, or combination thereof as described herein.

In certain embodiment, the thiosulfate is sodium thiosulfate (STS). In certain embodiment, the metabisulfite is sodium metabisulfite (SMB). In certain embodiment, the calcium phosphate tissue filler comprises calcium hydroxyapatite.

In certain embodiment, a volume ratio of the calcium phosphate tissue filler to the pharmaceutical composition ranges from about 4:1 to about 1:3. In certain embodiment, the volume ratio of the calcium phosphate tissue filler to the pharmaceutical composition is about 1:1, 1:2, 1:2.5 or any other volume ratios as described herein.

Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition. In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like.

The kits described herein contain one or more containers, which contain compounds, signaling entities, biomolecules and/or particles as described. The kits also contain instructions for mixing, diluting, and/or administrating the compounds. The kits also include other containers with one or more solvents, surfactants, preservative and/or diluents (e.g., saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known and are available in the literature. The solvent will depend on the compound and the mode of use or administration.

The kits comprise a carrier being compartmentalized to receive in close confinement one or more container such as vials, tubes, and the like, each of the container comprising one of the separate elements to be used in the method. For example, one of the container may comprise a positive control in an assay. Additionally, the kit may include containers for other components, for example, buffers useful in the assay.

In certain embodiments, the patient is informed that he or she should minimize strenuous activity and/or exposure of the treated area to extensive sun or heat exposure for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 36 hours, about 2 days, about 3 days, about 4 days, about 5 days, about 6 days, about 1 week, about 2 weeks, about 4 weeks, about 1 month, about 2 months, about 3 months, or longer than 3 months, after treatment, and/or until any initial swelling and redness has resolved.

### 6.10 Subjects

The terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human). The subject to be treated is in particular a mammal, preferably a human. The subject may be an adult or a child.

The following are examples of the present disclosure and are not to be construed as limiting.

### 7. EXAMPLES

### 7.1 Example 1

In a prospective, single-center clinical trial, 12 porcine samples (pieces of fresh pork shoulder) were injected with Radiesse®, with volumes ranging from 0.4 to 0.8 mL.

The samples were then randomized to three treatment groups: topical sodium metabisulfite (25% (w/v) SMB, formulated in petrolatum jelly: about 1-2 grams SMB applied topically to each site on the skin, 25% (w/v) sodium metabisulfite in 120 ml gel) application with occlusion, intralesional injection of about 0.2 mL of sodium thiosulfate solution (STS, concentration 12.5 g/50 mL, i.e., 0.25 g/mL or 25% (w/v)), or both. The volume ratio of Radiesse:STS solution ranged from 2:1 to 4:1. In the control group, samples were not treated after the Radiesse® injection.

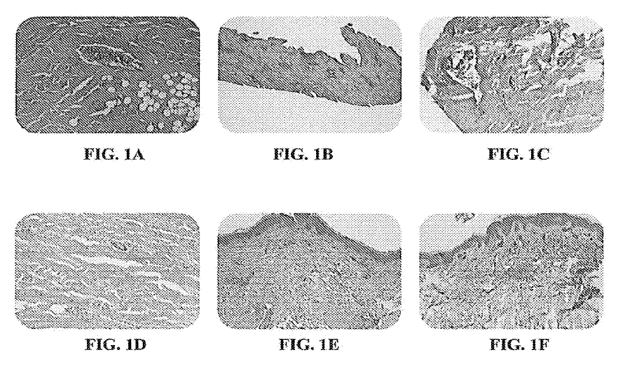

Twenty-four hours after treatment, a 4-mm punch biopsy was obtained and processed for histology to evaluate the presence, absence, or degradation of calcium hydroxyapatite (FIGS. 1A-1D). A blinded, board-certified dermatopathologist read the samples and determined the amount of calcium present in each sample.

In addition to punch biopsies, other possible methods to quantify the Radiesse® that still remains in a patient includes palpating for nodules. In addition, x-ray may be used.

### Results

Twenty-four hours after the STS and/or SMB treatment, intralesional STS injection combined with topical SMB, or intralesional STS injection alone, completely dissolved calcium hydroxyapatite in the samples. Topical SMB treatment reduced, but did not entirely clear calcium hydroxyapatite in tissue samples. The control samples contained easily identifiable calcium depositions. Table 1 lists the percentages of calcium hydroxyapatite reduced (e.g., degraded) 24 hours after various treatments.

TABLE 1

| | | STS | SMB | STS & SMB |
|---|---|---|---|---|
| | Control | treatment | treatment | treatment |
| % of calcium | 0% | 100% | ~50% | 100% |
| hydroxyapatite degraded | | | | |
| 24 hrs after treatment | | | | |

### 7.2 Example 2

Human skin samples (two living skin samples taken from living tissue in each group) were injected with 0.4 mL Radiesse®.

The samples were then randomized to three treatment groups: topical sodium metabisulfite (25% (w/v) SMB, formulated in petrolatum jelly, about 1-2 grams SMB applied topically to each site on the skin) application with occlusion, intralesional injection of of about 0.4-0.8 mL sodium thiosulfate solution (STS, concentration 12.5 g/50 mL i.e., 0.25 g/mL), or both. In the control group, samples were not treated after the Radiesse® injection.

Twenty-four hours after treatment, a 4-mm punch biopsy was obtained and processed for histology to evaluate the presence, absence, or degradation of calcium hydroxyapatite (FIGS. 1E-1F). A blinded, board-certified dermatopathologist read the samples and determined the amount of calcium present in each sample.

### Results

Twenty-four hours after the STS and/or SMB treatment, intralesional STS injection combined with topical SMB, or intralesional STS injection alone, completely dissolved calcium hydroxyapatite in the samples. Topical SMB treatment reduced, but did not entirely clear calcium hydroxyapatite in tissue samples. The control samples contained easily identifiable calcium depositions.

TABLE 2

| | | STS | SMB | STS & SMB |
|---|---|---|---|---|
| | Control | treatment | treatment | treatment |
| % of calcium | 0% | 100% | ~50% | 100% |
| hydroxyapatite degraded | | | | |
| 24 hrs after treatment | | | | |

Of note, observations of human tissue treated with intralesional injection of sodium thiosulfate and/or topical sodium metabisulfite illustrated no apparent deleterious effects on collagen or surrounding structures.

### Conclusions

This experiment demonstrates the reversibility of the Radiesse® calcium hydroxyapatite filler. This is an important landmark for patient safety and satisfaction, and serves as a potential model for treatment of other disorders of calcification, such as systemic calcium hydroxyapetite deposition disease.

### 7.3 Example 3

In the prospective histology study, 3 patients received Radiesse® injections (12-20 injections/patient) followed by intralesional STS injection (STS, concentration 12.5 g/50 mL, i.e., 0.25 g/mL) in the abdomen/arm prior to abdominoplasty/brachioplasty. Patients received various Radiesse:STS solution volume ratios ranging from 1:1 to 1:2.5, including patient 1: 0.5 mL CaHA and 0.5 mL STS per site (volume ratio of CaHA:STS is 1:1); patient 2: 0.2 mL CaHA and 0.5 mL STS per site (volume ratio of CaHA:STS is 1:2.5); patient 3: 0.15 mL CaHA and 0.3 mL STS per site (volume ratio of CaHA:STS is 1:2). Tissue was harvested about 1-2 h after STS injection, and a 4-mm punch biopsy was assessed for the presence of CaHA (using hematoxylin-eosin sections).

### Case 1—Female Abdominoplasty

12 samples
4 syringes of Radiesse=6.0 mL
0.5 mL CaHA per injection site

### 6 mL of STS

0.5 mL STS per site
1:1 CaHA/STS volume ratio
1.5 hours until harvest and punch biopsy

### Case 2—Female Abdominoplasty

16 samples
2 syringes of Radiesse=3.0 mL
0.2 mL CaHA per injection site

### 8 mL of STS

0.5 mL STS per injection
1 hour until harvest and punch biopsy
1:2.5 CaHA/STS volume ratio

### Case 3—Female Brachioplasty

20 samples
2 syringes of Radiesse=3.0 mL
0.15 mL CaHA per injection site

### 6 mL of STS

0.3 mL STS per injection site
1:2 CaHA/STS volume ratio
2 Hours until harvest and punch biopsy Findings from the above study are complemented by a case report. A female (33 years of age) presented with nodules (both deep and superficial) near the chin/jawline 3-months post Radiesse®/CaHA treatment. At visit 1, deep CaHA nodules were injected using a 32-gauge half inch needle with STS (STS, concentration 12.5 g/50 mL, i.e., 0.25 g/mL; 2 injections; 0.3 mL total). At visit 2 (about 3-4 weeks after visit 1), the patient received subsequent injections using a 32-gauge half inch needle on a luer lock syringe of STS (0.3 mL total) to superficial CaHA nodules.

### Results

At 1-2 h post-STS injection, CaHA was markedly dissolved in all human tissue samples in the histologic analyses (e.g., CaHA was decreased no less than 70%, no less than 75%, no less than 80%, no less than 85%, no less than 90%, no less than 95%, or more). All CaHA:STS ratios were associated with similar levels of CaHA reduction. Retention of CaHA spheres was observed in control samples without the STS administration.

In the case report of the 33-year-old female, the first injection of STS resulted in an immediate softening of the deep nodules. The patient reported progressive reduction in nodule sizes over the next 4 days or so, with a concomitant reduction in erythema. At visit 2, superficial nodules injected with STS softened and became semi-fluctuant within 1-2 min. The patient reported a burning sensation upon injection of STS at both visits (about 5-6 on a 10-point pain scale); however, the sensation resolved within about 1 min after immediate ice application.

### Conclusions

The results demonstrate reversibility of CaHA with STS injection in both controlled settings and in a patient case report of CaHA-related nodules.

### 7.4 Example 4

A filler composition comprising CaHA and HA (hyaluronic acid) was used in a subject. 0.4 ml of CaHA was mixed with 0.4 ml of a highly cohesive HA filler and injected into the volar surface of a human forearm. 0.4 ml of intralesional (IL) STS was injected intralesionally into the treated area. 4 hours after treatment, a 5-mm punch biopsy was obtained. H &E was used to evaluate the presence, absence, or degradation of calcium hydroxyapatite and hyaluronic acid. A blinded, board-certified dermatopathologist read the samples and determined the amount of calcium present in each sample.

### Results

Twenty-four hours after intralesional STS injection, calcium hydroxyapatite completely dissolved in the sample. In other words, the amount of the calcium phosphate tissue filler is reduced about 100% about 24 hours after administration of the pharmaceutical composition comprising STS. Even for a mixture of CaHA and HA, STS was still able to completely dissolve CaHA at a reduced time period of 4 hours. Wispy blue collections suggestive of HA was still observed in the sample tissue. Again, human tissue treated with intralesional injection of sodium thiosulfate illustrated no apparent deleterious effects on collagen or surrounding structures.

### Conclusions

The results demonstrate reversibility of fillers comprising CaHA and hyaluronic acid using STS injection. CaHA was completely dissolved even in the presence of hyaluronic acid making STS the ideal treatment for reducing CaHA-containing fillers.

### 8. EXEMPLARY SYSTEMS AND METHODS ARE SET OUT IN THE FOLLOWING ITEMS

Item 1. A method of reducing the amount of calcium phosphate tissue filler implanted in a subject, comprising the step of administering to the subject an effective amount of a pharmaceutical composition comprising a thiosulfate, a metabisulfite, or a combination thereof.
Item 2. The method of any of the preceding items, wherein the thiosulfate is sodium thiosulfate (STS).
Item 3. The method of any of the preceding items, wherein the pharmaceutical composition is administered to the subject through intralesional injection.
Item 4. The method of any of the preceding items, wherein the pharmaceutical composition comprises about 25% (w/v) sodium thiosulfate.
Item 5. The method of any of the preceding items, wherein the metabisulfite is sodium metabisulfite (SMB).
Item 6. The method of any of the preceding items, wherein the pharmaceutical composition is administered topically to the subject.
Item 7. The method of any of the preceding items, wherein the pharmaceutical composition comprises about 25% (w/v) sodium metabisulfite.
Item 8. The method of any of the preceding items, wherein the calcium phosphate is calcium hydroxyapatite.
Item 9. The method of any of the preceding items, wherein the amount of the calcium phosphate tissue filler is reduced at least 50% about 24 hours after administration of the pharmaceutical composition.
Item 10. The method of any of the preceding items, wherein the amount of the calcium phosphate tissue filler is reduced at least 80% about 24 hours after administration of the pharmaceutical composition.
Item 11. The method of any of the preceding items, wherein the amount of the calcium phosphate tissue filler is reduced at least 90% about 24 hours after administration of the pharmaceutical composition.
Item 12. The method of any of the preceding items, wherein the volume ratio of the tissue filler to the pharmaceutical composition ranges from about 4:1 to about 1:3.
Item 13. The method of any of the preceding items, wherein the volume ratio of the tissue filler to the pharmaceutical composition is about 1:1, 1:2 or 1:2.5.
Item 14. The method of any of the preceding items, wherein the calcium phosphate tissue filler is implanted into the subject in a plastic and/or reconstructive surgery.
Item 15. The method of any of the preceding items, wherein the calcium phosphate tissue filler is for hand augmentation.
Item 16. The method of any of the preceding items, wherein the calcium phosphate tissue filler is for hard or soft tissue augmentation, regeneration, and/or repair.
Item 17. The method of any of the preceding items, wherein the calcium phosphate tissue filler is for skin contour deficiencies.
Item 18. The method of any of the preceding items, wherein the skin contour deficiencies comprise frown lines, worry lines, wrinkles, crow's feet, marionette lines, stretch marks, scars, or combinations thereof.
Item 19. The method of any of the preceding items, wherein the subject is a human. Item 20. A method of treating a calcification disorder in a subject, comprising the step of administering to the subject an effective amount of a pharmaceutical composition comprising a thiosulfate, a metabisulfite, or a combination thereof.
Item 21. The method of any of the preceding items, wherein the thiosulfate is sodium thiosulfate (STS).
Item 22. The method of any of the preceding items, wherein the pharmaceutical composition comprises about 25% (w/v) sodium thiosulfate.
Item 23. The method of any of the preceding items, wherein the metabisulfite is sodium metabisulfite (SMB).
Item 24. The method of any of the preceding items, wherein the pharmaceutical composition comprises about 25% (w/v) sodium metabisulfite.
Item 25. The method of any of the preceding items, wherein the pharmaceutical composition is administered topically.
Item 26. The method of any of the preceding items, wherein the pharmaceutical composition is administered systemically.
Item 27. The method of any of the preceding items, wherein the calcification disorder is a cutaneous calcification or a subcutaneous calcification.
Item 28. The method of any of the preceding items, wherein the calcification disorder is calcium hydroxyapatite depositions resulting from implantation of calcium phosphate tissue filler.
Item 29. The method of any of the preceding items, wherein the calcification disorder is a systemic calcium hydroxyapatite deposition disease.
Item 30. The method of any of the preceding items, wherein the calcification disorder is a cardiovascular calcification.
Item 31. Use of a thiosulfate, a metabisulfite, or a combination thereof, in the manufacture of a medicament for reducing an amount of calcium phosphate tissue filler implanted in a subject.
Item 32. Use of a thiosulfate, a metabisulfite, or a combination thereof, in the manufacture of a medicament for treating a calcification disorder in a subject.
Item 33. The use of any of the preceding items, wherein the thiosulfate is sodium thiosulfate (STS).
Item 34. The use of any of the preceding items, wherein the medicament is administered to the subject through intralesional injection.
Item 35. The use of any of the preceding items, wherein the medicament comprises about 25% (w/v) sodium thiosulfate.
Item 36. The use of any of the preceding items, wherein the metabisulfite is sodium metabisulfite (SMB).
Item 37. The use of any of the preceding items, wherein the medicament is administered topically to the subject.
Item 38. The use of any of the preceding items, wherein the medicament comprises about 25% (w/v) sodium metabisulfite.
Item 39. The use of any of the preceding items, wherein the calcium phosphate is calcium hydroxyapatite.
Item 40. The use of any of the preceding items, wherein the amount of the calcium phosphate tissue filler is reduced at least 50% about 24 hours after administration of the medicament. Item 41. The use of any of the preceding items, wherein the amount of the calcium phosphate tissue filler is reduced at least 80% about 24 hours after administration of the medicament.
Item 42. The use of any of the preceding items, wherein the amount of the calcium phosphate tissue filler is reduced at least 90% about 24 hours after administration of the medicament.
Item 43. The use of any of the preceding items, wherein the volume ratio of the tissue filler to the medicament ranges from about 4:1 to about 1:3.
Item 44. The use of any of the preceding items, wherein the volume ratio of the tissue filler to the medicament is about 1:1, 1:2 or 1:2.5.
Item 45. The use of any of the preceding items, wherein the calcium phosphate tissue filler is implanted into the subject in a plastic and/or reconstructive surgery.
Item 46. A pharmaceutical composition comprising a thiosulfate, a metabisulfite, or a combination thereof, for use in reducing an amount of calcium phosphate tissue filler implanted in a subject.
Item 47. A pharmaceutical composition comprising a thiosulfate, a metabisulfite, or a combination thereof, for use in treating a calcification disorder in a subject.
Item 48. The pharmaceutical composition of any of the preceding items, wherein the thiosulfate is sodium thiosulfate (STS).
Item 49. The pharmaceutical composition of any of the preceding items, wherein the pharmaceutical composition comprises about 25% (w/v) sodium thiosulfate.
Item 50. The pharmaceutical composition of any of the preceding items, wherein the metabisulfite is sodium metabisulfite (SMB).
Item 51. The pharmaceutical composition of any of the preceding items, wherein the pharmaceutical composition comprises about 25% (w/v) sodium metabisulfite.
Item 52. A pharmaceutical combination, preferably in a kit, comprising:
(a) a calcium phosphate tissue filler; and
(b) a pharmaceutical composition comprising a thiosulfate, a metabisulfite, or combination thereof.
Item 53. The pharmaceutical combination of any of the preceding items, wherein the pharmaceutical composition comprises about 25% (w/v) of the thiosulfate, metabisulfite, or combination thereof.
Item 54. The pharmaceutical combination of any of the preceding items, wherein the thiosulfate is sodium thiosulfate (STS).
Item 55. The pharmaceutical combination of any of the preceding items, wherein the metabisulfite is sodium metabisulfite (SMB).
Item 56. The pharmaceutical combination of any of the preceding items, wherein the calcium phosphate tissue filler comprises calcium hydroxyapatite.
Item 57. The pharmaceutical combination of any of the preceding items, wherein a volume ratio of the calcium phosphate tissue filler to the pharmaceutical composition ranges from about 4:1 to about 1:3.
Item 58. The pharmaceutical combination of any of the preceding items, wherein the volume ratio of the calcium phosphate tissue filler to the pharmaceutical composition is about 1:1, 1:2 or 1:2.5.

The scope of the present disclosure is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this disclosure. The citation and discussion of such references is provided merely to clarify the description of the present disclosure and is not an admission that any reference is prior art to the disclosure described herein. All references cited and discussed in this specification are incorporated herein by reference in their entirety. Variations, modifications and other implementations of what is described herein will occur to those of ordinary skill in the art without departing from the spirit and scope of the disclosure. While certain embodiments of the present disclosure have been shown and described, it will be obvious to those skilled in the art that changes and modifications may be made without departing from the spirit and scope of the disclosure. The matter set forth in the foregoing description and accompanying drawings is offered by way of illustration only and not as a limitation.

What is claimed is:

1. A method of reducing an amount of calcium phosphate tissue filler implanted in a subject, comprising the step of administering to the subject an effective amount of a pharmaceutical composition comprising a thiosulfate, a metabisulfite, or a combination thereof.

2. The method of claim 1, wherein the thiosulfate is sodium thiosulfate (STS).

3. The method of claim 2, wherein the pharmaceutical composition is administered to the subject through intralesional injection.

4. The method of claim 2, wherein the pharmaceutical composition comprises 25% (w/v) sodium thiosulfate.

5. The method of claim 1, wherein the metabisulfite is sodium metabisulfite (SMB).

6. The method of claim 5, wherein the pharmaceutical composition is administered topically to the subject.

7. The method of claim 5, wherein the pharmaceutical composition comprises 25% (w/v) sodium metabisulfite.

8. The method of claim 1, wherein the calcium phosphate is calcium hydroxyapatite.

9. The method of claim 1, wherein the amount of the calcium phosphate tissue filler is reduced at least 50% 24 hours after administration of the pharmaceutical composition.

10. (canceled)

11. The method of claim 1, wherein the amount of the calcium phosphate tissue filler is reduced at least 90% 24 hours after administration of the pharmaceutical composition.

12. The method of claim 1, wherein the volume ratio of the tissue filler to the pharmaceutical composition ranges from 4:1 to 1:3.

13. The method of claim 1, wherein the volume ratio of the tissue filler to the pharmaceutical composition is 1:1, 1:2 or 1:2.5.

14. The method of claim 1, wherein the calcium phosphate tissue filler is implanted into the subject in a plastic and/or reconstructive surgery.

15. The method of claim 1, wherein the calcium phosphate tissue filler is for hand augmentation.

16. The method of claim 1, wherein the calcium phosphate tissue filler is for hard or soft tissue augmentation, regeneration, and/or repair.

17. The method of claim 1, wherein the calcium phosphate tissue filler is for skin contour deficiencies.

18. The method of claim 17, wherein the skin contour deficiencies comprise frown lines, worry lines, wrinkles, crow's feet, marionette lines, stretch marks, scars, or combinations thereof.

19. The method of claim 1, wherein the subject is a human.

20. A method of treating a calcification disorder in a subject, comprising the step of administering to the subject an effective amount of a pharmaceutical composition comprising a thiosulfate, a metabisulfite, or a combination thereof.

21-51. (canceled)

52. A pharmaceutical combination, preferably in a kit, comprising:
(a) a calcium phosphate tissue filler; and
(b) a pharmaceutical composition comprising a thiosulfate, a metabisulfite, or combination thereof.

53-58. (canceled)
